(19) United States
(12) Patent Application Publication
(10) Pub. No.: US 2006/0116403 A1
(43) Pub. Date: Jun. 1, 2006

(54) Therapeutic target and uses thereof

(76) Inventor: Peter James Little, Victoria (AU)

Correspondence Address: Michael L Goldman;Nixon Peabody, Clinton Square, P O Box 31051, Rochester, NY, 14603, US

(21) Appl. No.: 10/515,393

(22) PCT Filed: May 20, 2003
(86) PCT No.: PCT/AU03/00608

§ 371 Date: Dec. 19, 2005

(30) Foreign Application Priority Data
May 20, 2002 (AU) PS 2430

(51) Int. Cl. A61K 31/44

(52) U.S. Cl. 514/352

(57) ABSTRACT

The present invention relates to the finding of a novel therapeutic target which is implicated in regulating glycosaminoglycan (GAG) length, and the use of this target particularly for regulating lipoprotein binding. More particularly the invention relates to the use of this target for methods of treating and preventing conditions associated with lipoprotein binding in tissues or blood vessels. More specifically, the invention resides in the use of the new target as a key biochemical target for the prevention and treatment of atherosclerosis and identifies useful therapeutic agents which may act on the target. Accordingly, in a first aspect of the present invention there is provided a method of controlling glycosaminoglycan (GAG) chain length in a cell, said method comprising modifying activation of c-Abl in the cell.

The present invention relates to the finding of a novel therapeutic target which is implicated in regulating glycosaminoglycan (GAG) length, and the use of this target particularly for regulating lipoprotein binding. More particularly the invention relates to the use of this target for methods of treating and preventing conditions associated with lipoprotein binding in tissues or blood vessels. More specifically, the invention resides in the use of the new target as a key biochemical target for the prevention and treatment of atherosclerosis and identifies useful therapeutic agents which may act on the target.

### BACKGROUND

Cardiovascular disease is the major cause of premature mortality in Western societies. The major underlying process is atherosclerosis leading to the formation of unstable plaques followed by thrombotic occlusion of the coronary arteries, myocardial ischemia, heart failure and death. Increasing rates of obesity resulting from increasingly sedentary lifestyles and dietary factors are underpinning an epidemic of (Type 2) diabetes, a state of multiple dysmetabolic factors which underlie an increased rate of cardiovascular disease by at least two-fold. Rates of cardiovascular disease were declining in the second half of last century but alarmingly the decline has slowed or reversed in recent years.

The critical events in the development and rupture of atherosclerotic plaques are unknown. Current therapies have been developed to target presently identified “risk factors” specifically hypertension, hypercholesterolemia and hypertriglyceridemia and, in the presence of diabetes, hyperglycemia. There has been evidence that some agents including the statins for treating hypercholesterolemia and angiotensin converting enzyme inhibitors for the treatment of hypertension may have some direct vascular actions and these have been termed “pleiotropic”. Although some of these agents are effective in reducing cardiovascular disease, the preferred therapeutic modality would be to identify the critical atherogenic events and develop agents specifically targeted at those mechanisms.

One prominent hypothesis for the origin of atherogenesis, known as “response to retention”, states that the sub-endothelial binding and retention of atherogenic lipoproteins by matrix molecules is a critical step. Low density lipoproteins bind to the glycosaminoglycan (GAG) chains covalently bound to the core proteins of proteoglycans. The size of these GAG chains is regulated and considerable evidence is emerging to suggest that the binding of low density lipoproteins (LDL) is related to the size of the GAG chains such that longer chains are “stickier” and vice versa. The mechanisms and processes controlling GAG length are presently unknown but represent a prime target for the development of therapeutic agents which may reduce GAG length and consequentially reduce LDL binding and retention in the vessel wall, potentially greatly reducing the initiation and progression of atherosclerosis.

Growth factors and atherogenic stimuli cause elongation of GAG chains and promote LDL binding. Several classes of drugs including calcium antagonists and glucosamine have been shown to reduce the size of GAG chains and reduce LDL binding. However, there is no knowledge of the critical signaling pathways or the mechanisms controlling the biosynthetic pathways which determine the ultimate length of GAG chains.

Although several atherogenic stimuli are known to stimulate the elongation of GAG chains on proteoglycans and increase LDL binding there is no example of how this process can be targeted successfully with a potent and specific agent with the potential to be developed as an agent for human therapeutic use. There are potentially two targets for reducing the size of proteoglycans—the mechanisms actually responsible for the elongation process and the signaling mechanism such as that for growth factors which stimulate elongation. Little is known of the biochemistry of GAG elongation in cells such as vascular smooth muscle cells (VSMC).

Platelet-derived growth factor (PDGF) stimulates the production and increases the size of proteoglycans, with the latter response due to elongation of the GAG chains. PDGF also stimulates the expression of mRNA for proteoglycan core proteins, specifically the large chondroitin sulfate proteoglycan, versican. PDGF receptors are members of the Type III receptor tyrosine kinase family. Upon ligand engagement receptor activation initiates autophosphorylation and subsequent signaling cascades leading to activation of vascular smooth muscle cells and the expression of functions and properties associated with atherogenesis such as chemotaxis, proliferation and increased matrix production. PDGF stimulates proteoglycan biosynthesis increasing the expression of versican mRNA and core protein and stimulating the elongation of GAG chains. Although it has not yet been expressly demonstrated, by analogy with the effect of growth factors such as transforming growth factor (TGF)β it is likely larger GAG chains of proteoglycans produced by vascular smooth muscle cells stimulated by PDGF would also be associated with increased binding of LDL. The isoflavinoid, genistein, is a “broad spectrum” tyrosine kinase inhibitor which inhibits PDGF-stimulated proliferation and total proteoglycan biosynthesis by vascular smooth muscle cells. Proteoglycan biosynthesis is assessed as ³⁵S-radiosulfate incorporation into GAG chains. Reduced incorporation of ³⁵S-radiosulfate can therefore arise from reduced core protein synthesis and thus less GAG initiation sites or by reduction in the length of the GAG chains. Genistein inhibits the synthesis of proteoglycan core proteins, specifically the large chondroitin sulfate proteoglycan versican, and has no effect on GAG length. From these data it was concluded that tyrsoine kinases do not play a role in the signaling pathways that control the length of the GAGs on proteoglycans. However, recent evidence has suggested that PDGF may elicit signaling which is directly or indirectly additional to the signaling through stimulation of the phosphorylation of its receptor tyrosine kinase.

Accordingly, this invention attempts to identify a therapeutic target which can regulate GAG length and which can be used to form the basis for rational therapy to prevent or treat lipoprotein associated conditions.

### SUMMARY

In a first aspect of the present invention there is provided a method of controlling glycosaminoglycan (GAG) chain length in a cell, said method comprising modifying activation of c-Abl in the cell.

Applicants have found a key biochemical target for regulating the length of GAG in a cell. The cytosolic, non-receptor tyrosine kinase Abelson oncogene c-Abl is a major signalling pathway for both basal and PDGF mediated GAG.

The oncogene may be modified by inhibition or activation to increase or decrease GAG length and thereby modify lipoprotein binding in a cell or tissue.

A preferred inhibitor of c-Abl activation is imatinib or an equivalent thereof.

In yet another aspect of the invention, there is provided a method of treating atherosclerosis in a patient, said method comprising administering a therapeutically effective amount of a c-Abl inhibitor or equivalent to the patient.

By regulating the length of GAG chains on proteoglycans in cells and thereby regulating lipoprotein binding, the initiation and progression of atherosclerosis may be controlled. Inhibition of c-Abl will affect the GAG length and lipoprotein binding.

In a preferred aspect of the invention, there is provided a method of reducing a risk for atherosclerosis, said method comprising

identifying a risk factor for atherosclerosis; and

administering an amount of a c-Abl inhibitor to the patient to prevent progression of atherosclerosis.

Some patients show a propensity for atherosclerosis. These patients may be identified by showing high risk factors such as high blood pressure, high cholesterol and/or diabetes. These factors are closely associated with atherosclerosis. By identifying these patterns early, these patients may be prevented from progressing to atherosclerosis by administering a low dose or an effective dose of a c-Abl inhibitor such as imatinib, which can prevent progression of the disease. The amount may also be used to halt, slow down or maintain the progression and prevent accelerated progression of the disease.

In yet another aspect of the present invention, there is provided a composition for use in treating atherosclerosis, said composition comprising a therapeutically effective amount of a c-Abl inhibitor or equivalent thereof and a pharmaceutically acceptable carrier.

### FIGURES

FIG. 1 shows the effects of tyrosine kinase inhibitors on total proteoglycan production in human VSMCs. Serum derived cells were metabolically labeled with ³⁵S-sulfate for 24 h in the presence of imatinib (1 μM), genistein (100 μM) or daidzein (100 μM) and the proteoglycans produced and secreted into the medium were isolated and quantitated as shown. *P<0.05; **P<0.01.

FIG. 2 shows analysis of the size of proteoglycans produced in cells treated with imatinib, genistein and daidzein and metabolically labeled with ³⁵S-sulfate. Secreted proteoglycan produced by human vascular smooth muscle cells were isolated and purified by Sepharose anion exchange chromatography and subjected to SDS-PAGE (upper image). The proteoglycans were subjected to treatment with sodium cyanoborohydride and alkali to release the GAG chains from the core protein and the size analysis of the free chains by SDS-PAGE is shown (lower image).

FIG. 3 shows effects of imatinib, genistein and daidzein on the biosynthesis of artificial GAG chains synthesized on exogenous xyloside. Cells metabolically labeled with ³⁵S-sulfate and supplemented with xyloside (0.5 mM) synthesize and secrete proteoglycans and xyloside-initiated GAG chains. The effect of the tyrosine kinase inhibitors on the quantitative production of total ³⁵S-sulfate incorporation (upper panel) and the size of the resultant products is shown (lower panel). *P<0.05; **P<0.01.

FIG. 4 shows the concentration dependence of the inhibition of ³⁵S-sulfate into proteoglycans by imatinib. Serum derived cells were metabolically labeled with ³⁵S-sulfate for 24 h in the presence of multiple concentrations of imatinib (1 nM to 10 μM) and platelet-derived growth factor (50 ng/mL as indicated). The proteoglycans produced and secreted into the medium were isolated and quantitated described above.

FIG. 5 shows effect growth factor stimulation and tyrosine kinase inhibition on the de novo production of total proteoglycan core proteins. The cells were metabolically labeled with ³⁵S-methionine/cysteine and the proteoglycans were isolated from the cultured media by the CPC precipitation method which is specific for proteoglycans. *P<0.05; **P<0.01: ***P<0.001.

FIG. 6 shows low density lipoprotein binding (LDL) capacity of proteoglycans secreted by human vascular smooth muscle cells stimulated by PDGF in the presence of tyrosine kinase inhibitors with different effects on GAG length. Total proteoglycans were isolated and purified from control cells or PDGF stimulated cells treated with imatinib or genistein and applied to individual LDL affinity columns prepared by protecting proteoglycan binding sites with heparin. Data shows the total bound proteoglycans which were released from the affinity columns by 1M NaCl.

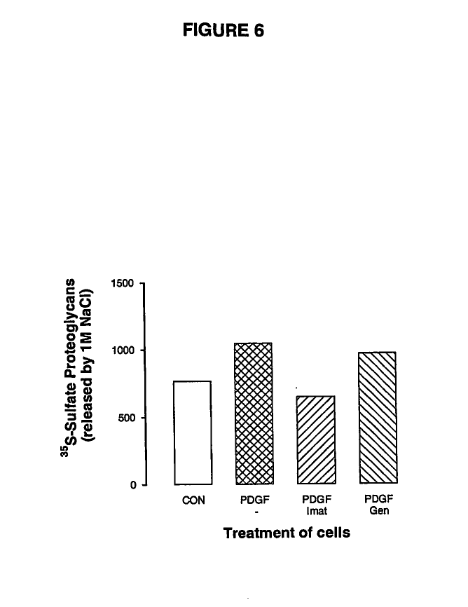

FIG. 7 shows a scheme showing the pathways of platelet-derived growth factor (PDGF) signaling in vascular smooth muscle cells and the novel pathway controlling glycosaminoglycan (GAG) length of proteoglycans. Imatinib inhibits both PDGF receptor and c-Abl tyrosine kinases whereas genistein inhibits only PDGF receptor tyrosine kinase. c-Abl is central to the pathway of glycosaminoglycan (GAG) elongation on proteoglycans and hence inhibition of c-Abl represents a target for reduction in GAG size and reduces lipoprotein binding (see FIG. 6). The scheme indicates that PDGF activates c-Abl via the PDGF receptor but not involving the tyrosine phosphorylation of the PDGF receptor since c-Abl is activated in genistein treated cells in which receptor phosphorylation is inhibited. “inh”: inhibition. PDGFR is the PDGF receptor.

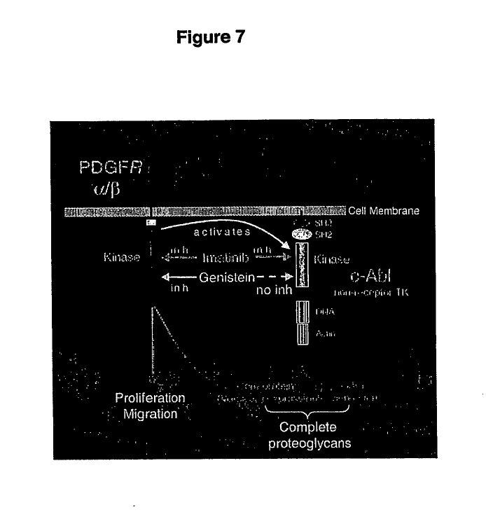

FIG. 8 shows direct measurement of cAbl kinase activity and the effect of various compounds relevant to the present discovery. The data shows the extreme potency of imatinib and the considerably reduced activity for other compounds (genistein and herbimycin) which are considered to be broad spectrum Protein Tyrosine Kinase (PTK) inhibitors. Daidzein is the “inactive analogue of genistein (inactive for kinases towards which genistein shows inhibitory activity). Most inhibitors inhibit by interacting with the ATP binding of the kinase and it is not surprising that at very high concentrations in this direct biochemical assay that compounds such as genistein do show a small amount of inhibitory activity (See FIG. 9 for effect in a whole cell system).

FIG. 9 shows the effect of the broad spectrum PTK inhibitor, herbimycin, on radiosulphate incorporation (FIG. 9A; upper panel) and GAG size (FIG. 9B; lower panel) on proteoglycans produced by vascular smooth muscle cells. Herbimycin which shows no inhibitory activity towards cAbl (see FIG. 8) did not inhibit radiosulphate incorporation into proteoglycans nor shorten the GAGs on the PGs and this is consistent with the contention that the GAG shortening occurs via (inhibition of) cAbl kinase activity.

FIG. 10 shows the inhibition of proliferation of K562 cells. Proliferation of these tumour cells is driven by cAbl and this is the target of imatinib used for Chronic Myeloid Leukemia. Proliferation was assessed in the presence of several compounds used in the current project. Imatinib was a potent inhibitor of proliferation, but genistein showed little or no inhibitory activity indicating that it does not inhibit cAbl in a whole cell based assay consistent with the observation that genistein does not shorten GAGs on proteoglycans and that this is consistent with imatinib acting via inhibition of cAbl.

FIG. 11 shows that inhibition of cAbl shortens GAGS on PGs produced by saphenous vein vascular smooth muscle cells. All earlier data has been based on experiments with Internal Mammary Artery derived vascular smooth muscle cells. Saphenous vein samples were obtained from Cardiac Theatres at the Alfred Hospital and developed vascular smooth muscle cell cultures for experiments. The data shows that imatinib inhibits radiosulphate incorporation into proteoglycans produced and secreted into the culture media by the cells (FIG. 11A; upper panel) and that this is associated with a reduction in the size of the proteoglycans as evidenced by increased electrophoretic mobility on SDS-PAGE. (FIG. 11B; lower panel).

FIG. 12 shows size exclusion chromatography to determine the size of the proteoglycans (FIG. 12A; upper panel) and the effect of growth factor and imatinib treatment (FIG. 12B; lower panel). Proteoglycans radiolabeled with ³⁵S-sulphate were isolated from the cell culture media and isolated by DEAE sepharose ion exchange chromatography. The glycosaminoglycan chains from the proteoglycans were chemically released from the proteoglycan core proteins and subjected to size exclusion chromatography on sepharose 6B columns. Sizes (Kav values are given in the text). (We provided these numbers if you wish to use them in the text or a table.)imatinib decreased the size of control GAGs and also prevented the size increase elicited by PDGF.

FIG. 13 shows the use of gene silencing to study the role of cAbl in determination of GAG length on vascular smooth muscle cell proteoglycans. The data shows that an siRNA directed to cAbl (and thus the elimination of cAbl from the cells) mimics the action of imatinib consistent with the conclusion that cAbl is the critical signalling kinase in the PDGF mediated GAG elongation that would be associated with LDL binding and retention in the process of atherogenesis. This figure shows the effects on radiolabeled proteoglycans secreted into the culture media (FIG. 13A; upper panel) and on proteoglycans remaining in the cell layer (FIG. 13B; lower panel).

FIG. 14 shows the incorporation of ³⁵S-methionine/cysteine into the core proteins of proteoglycans. Cells were radiolabeled with ³⁵S-methionine/cysteine and proteoglycans were quantified by the CPC precipitation method. The data shows a small stimulatory effect of PDGF and the inhibitory effect of imatinib which in this case is based on its ability to inhibit the PDGF receptor. The aim of this experiment is to produce radiolabeled proteoglycans which are labelled on their core proteins to remove the confounding effect which results from studying the binding of ³⁵S-sulphate labelled GAG chains on proteoglycans where the label is part of the chains, when the chains are considered to determine the binding.

FIG. 15A shows Gel Mobility Shift Analysis of the binding of Control Core Protein (³⁵S-Methionine/Cysteine) labelled proteoglycans to human LDL. Proteoglycans secreted into the culture media were isolated and purified and subjected to GSMA with various concentrations of human LDL. The upper panel shows the Gel and the lower panel shows the graphical presentation of the data. The data shows that proteoglycans produced by cells stimulated with PDGF and treated with imatinib show lower affinity binding to LDL

FIG. 15B show Gel Mobility Shift Analysis of the binding of PDGF stimulated Core Protein (³⁵S-Methionine/Cysteine) labelled proteoglycans to human LDL. Proteoglycans secreted into the culture media were isolated and purified and subjected to GSMA with various concentrations of human LDL. The upper panel shows the Gel and the lower panel shows the graphical presentation of the data. The data shows that proteoglycans produced by cells stimulated with PDGF and treated with imatinib show lower affinity binding to LDL.

### DESCRIPTION OF THE INVENTION

In a first aspect of the present invention there is provided a method of controlling glycosaminoglycan (GAG) chain length in a cell, said method comprising modifying activation of c-Abl in the cell.

Applicants have found a key biochemical target for regulating the length of GAG in a cell. The cytosolic, non-receptor tyrosine kinase Abelson oncogene c-Abl is a major signalling pathway for both basal and PDGF mediated GAG elongation.

c-Abl is also involved in cancer through a role for the Bcr-c-Abl oncoproteins in the nucleus in cell growth and apoptosis and plays a key role in the development of several forms of human leukemias. However c-Abl has also been shown to be present and active in the cytoplasm and the membrane of cells where it is associated with actin filaments. The membrane pool of c-Abl in NIH-3T3 fibroblasts is activated by PDGF. PDGF activates Src which activates c-Abl but the activation of c-Abl by growth factors may occur through Src-dependent and Src-independent pathways. An actin-dependent “ruffling” response in fibroblasts is lost in c-Abl −/− cells and restored in cells transfected with c-Abl. c-Abl has recently been identified in vascular smooth muscle cells in the A10 cell line derived from rat aorta. c-Abl is hyperphosphorylated by serum leading to the formation of a larger molecular species.

The method of controlling the GAG chain length may include inhibiting, activating or stimulating processes involved in GAG elongation resulting in increasing or decreasing GAG chain length.

The term “modifying activation of c-Abl” as used herein means to change the activation either by increasing or decreasing the activation of c-Abl. The oncogene may be activated to increase GAG chain length or it may be inhibited to decrease GAG chain length or prevent elongation of the GAG chains. The present invention includes full and partial inhibition of the GAG elongation process. The extent to which the elongation processes are inhibited or stimulated will depend upon the effect placed on modifying activation c-Abl to manifest in an altered GAG chain length.

The methods of modifying activation to inhibit or stimulate c-Abl may include direct or indirect activation of c-Abl. Any pathways involved in the activation of upstream or downstream modifiers are included in this invention. The activation of c-Abl may be increased or decreased compared to un-activated c-Abl.

Modifying the activation of c-Abl may be achieved using antagonists, inhibitors, mimetics, or derivatives of c-Abl. The term “antagonist” or “inhibitor” as used herein, refers to a molecule which when bound to c-Abl blocks or modulates the activation of c-Abl. Antagonists or inhibitors may include proteins, nucleic acids, carbohydrates, antibodies, or any molecules including ligands which can bind to c-Abl either directly or indirectly to induce an effect on c-Abl. Other “antagonists” or “inhibitors” include a range of rationally designed, synthetic inhibitors, generally based on direct inhibitors of c-Abl.

Other direct methods to achieve a modified activation of c-Abl may include, but not limited to, knockout technology, antisense technology, triple helix technology, targeted mutation, gene therapy or regulation by-agents acting on transcription. Indirect methods include targeting upstream or downstream regulators such as but not limited to regulators of cytosolic tyrosine kinases and PDGF receptors.

Throughout the description and claims of this specification, the word “comprise” and variations of the word, such as “comprising” and “comprises”, is not intended to exclude other additives, components, integers or steps.

In a preferred embodiment there is provided a method of controlling GAG chain length by reducing GAG chain elongation in a cell, said method comprising reducing activation of c-Abl in the cell.

Reducing extension of the GAG chains results from inhibiting the process of GAG extension on the proteoglycan core protein. Inhibition may be induced by the use of specific c-Abl inhibitors.

Preferably, the c-Abl inhibitor is imatinib or an equivalent. An equivalent of imatinib as used herein means a compound which behaves in a similar manner to imatinib so as not to be able to distinguish between the two compounds. The equivalent should preferably target c-Abl at the same sites as imatinib and therefore induce the same outcome as imatinib.

The inhibitor imatinib is a 2-phenyl aminopyridine derivative that has recently been introduced for the treatment of human chronic myeloidleukemia (CML) in which the c-Abl is constitutively activated. It has inhibitory effects on the growth of cancer cells in which c-Abl is activated but little effect on non-transformed cells. Imatinib, like genistein, inhibits PDGF receptor kinase, but in addition it inhibits c-Abl and inhibits endogenous and PDGF-stimulated GAG chain elongation in the cell and hence reduces the size of proteoglycans in the cell.

The premise of the existing discovery in the present application arises from the action of imatinib to not only inhibit the PDGF receptor but also inhibit cAbl (kinase activity) in vascular smooth muscle and that this action is associated with glycosaminoglycan (GAG) shortening and reduced LDL binding and therefore the prevention of atherosclerosis. The major pharmacological agent available for the inhibition of Protein Tyrosine Kinases (PTKs) is genistein and this compound is colloquially considered to block all tyrosine kinases. Genistein however does not cause GAG shortening of vascular proteoglycans.

Accordingly, GAG chain length is preferably controlled by exposing the cell to an effective amount of imatinib or equivalent said amount being effective to inhibit or reduce GAG chain elongation.

The cell may be exposed to an amount of imatinib in the range of 1 nM to 10 μM, more preferably, imatinib is exposed to the cells at approximately 1 μM.

GAG chain length elongation may be reduced in any cells that contain c-Abl. Preferably the cells used herein include, but are not limited to cells selected from the group including endothelial cells, macrophages, fibroblasts and cells associated with atherosclerosis. Preferably the cells are muscle cells, more preferably the cells are vascular smooth muscle cells.

GAG chain length may be monitored by any methods available to the skilled addressee which can compare a reduction in proteoglycan size over time. Electrophoretic mobility or size exclusion chromatography are preferred.

In another aspect of the present invention there is provided a method of controlling lipoprotein binding in a cell or tissue, said method comprising controlling GAG chain length in the cell by modifying activation of c-Abl in the cell.

Large GAG chains show enhanced lipoprotein binding because it is considered that longer GAG chains are “stickier”. Without being limited by theory, it is postulated that by reducing GAG chain length, the amount of lipoprotein binding can be reduced.

In a preferred embodiment, there is provided a method of reducing lipoprotein binding in a cell or tissue, said method comprising reducing GAG length in the cell by reducing activation of c-Abl in the cell.

A preferred method of reduction is by inhibition, preferably by inhibition of c-Abl which has been found to be a target point for the control of GAG chain length. Most preferably, the inhibitor of c-Abl is imatinib or an equivalent thereof. However, any compound as described above that targets c-Abl either directly or indirectly may be used.

An imatinib-sensitive pathway regulates the length of GAG chains on proteoglycans produced by human vascular smooth muscle cells and the consequences for lipoprotein binding. Genistein and imatinib inhibit basal and PDGF-stimulated proteoglycan biosynthesis, however only imatinib reduces the size of the proteoglycans. Applicants have shown that the glycosaminoglycan chains released from proteoglycans produced by imatinib treated cells are reduced in size and the synthesis of artificial GAGs on exogenous xyloside confirms that imatinib specifically inhibits GAG elongation. Proteoglycans produced by imatinib but not genistein treated cells show reduced lipoprotein binding. Proteoglycan core protein biosynthesis stimulated by PDGF, a PDGF receptor phosphorylation-dependent process, is inhibited by both genistein and imatinib. The results show that imatinib inhibits both PDGF receptor tyrosine kinase autophosphorylation and c-Abl and that the latter is a key signaling pathway for GAG elongation. Hence, imatinib is a potential agent for reducing GAG size on vascular proteoglycans and is thus a new potential therapeutic agent to prevent lipid deposition and potentially treat atherogenesis.

To reduce lipoprotein binding, the cells may be exposed to 1 nM to 10 μM, more preferably, the cells are exposed to approximately 1 μm imatinib.

Binding of any lipoprotein may be reduced and the reduced binding of the lipoproteins may be due to a reduced binding of the proteoglycan to the externally located apolipoprotein associated with the lipoprotein. For instance, apolipoprotein B-100 (apo B-100) is associated with VLDL and its remnants, and apolipoprotein E (apo E) is associated with HDL subfractions. Apolipoprotein B-48 (apo B48) and Apo (a) also bind to proteoglycans. Accordingly via binding with apolipoprotein, and reduced GAG length, lipoprotein binding may be reduced. Preferably, the lipoproteins in which binding is reduced is selected from the group including LDL, VLDL and its remnants and HDL.

Reaction of the activation of c-Abl may simply involve exposure of the cell to an inhibitor or modulator of c-Abl. The modulation of c-Abl need not be a direct inhibitor of c-Abl which results in a change in activation of c-Abl. Suitable modulators having indirect effects on c-Abl are discussed above.

Pre-plated cells may be exposed to the inhibitor or modulator of c-Abl in the media and exposed for a period sufficient to reduce GAG chain elongation and thereby reduce lipoprotein binding. Inhibition may be experienced within a 24 hour exposure from initial exposure to the inhibitor or modulator. The exposure may be enhanced by a compound which makes the inhibitor or modulator more permeable to the cell and to enhance the effect of delivery of the inhibitor or modulator to the cells. Alternatively, imatinib may be bound to a suitable carrier which enhances delivery of the compound to the cell.

Preferably, the cell is any cell which contains c-Abl. Preferably, the cells are selected from the group including, but not limited to, endothelial cells, macrophages, fibroblasts and cells associated with atherosclerosis. Proliferating cells have been found to make longer GAGs and hence, without being limited by theory, it is postulated that c-Abl is involved or activated in proliferating cells. Accordingly, cells which have high proliferating capacity are also preferred as targets to reduce lipoprotein binding by exposure to a c-Abl inhibitor.

Preferably the cells are muscle cells. More preferably, they are vascular smooth muscle cells. Cells in the new inner layer of blood vessels (“neo-intima”) are also preferred as are cells in early atherosclerotic plaques. These cells may have higher rates of proliferation and hence activated c-Abl. Such cells are considered to be useful targets for c-Abl inhibitors preferably by imatinib or equivalent thereof.

In yet another aspect of the invention, there is provided a method of treating atherosclerosis in a patient, said method comprising administering a therapeutically effective amount of a c-Abl inhibitor or equivalent to the patient.

Atherosclerosis is the major process underlying cardiovascular disease which is the largest cause of premature death in Western societies. Present therapies target “risk factors” because therapeutic agents for the causative biochemical mechanisms have not been developed. The “response to retention” hypothesis of atherogenesis proposes that vascular proteoglycans bind and retain atherogenic lipoproteins in the vessel wall—larger (glycosaminoglycan) GAG chains show enhanced lipoprotein binding and vice versa.

By regulating the length of GAG chains on proteoglycans in cells and thereby regulating lipoprotein binding, the initiation and progression of atherosclerosis may be controlled. Inhibition of c-Abl will affect the GAG length and lipoprotein binding.

The term “treatment” is used herein in its most broadest sense to include preventative treatments. In this respect, the treatment may include providing a c-Abl inhibitor at a dosage to prevent further progression of atherosclerosis.

Patients with atherosclerosis include patients with “confirmed” atherosclerosis as well as those patients having a high risk of atherosclerosis. For those patients with “confirmed” atherosclerosis, these may be “confirmed” patients established by accepted methods including coronary angiography or intravascular ultrasound. Patients with a high risk of atherosclerosis show standard indicators such as but not limited to, increased cholesterol, increased blood pressure and/or diabetes.

Preferably, the inhibitor of c-Abl is imatinib or an equivalent thereof. The cytosolic, non-receptor tyrosine kinase, c-Abl, which is inhibited by imatinib, is a major signaling pathway for both basal and PDGF mediated GAG chain elongation. Imatinib reduces the size of basal proteoglycans and completely inhibits the elongation stimulated by PDGF. The consequence of this reduction in size is a reduction in LDL binding which would, if operative in vivo, lead to a reduction in LDL accumulation in the vessel wall with the reduced lipid load assisting in the prevention and possibly reversal of atherosclerosis.

The “response to retention” hypothesis of atherogenesis invokes a role for proteoglycans in the binding and retention of atherogenic lipoproteins. Initial in vitro studies have shown that proteoglycans produced by VSCMs bind to LDL and the length of the GAG chains oh the proteoglycans directly determines LDL binding properties. However, although growth factors extend GAG chains and several agents reduce GAG length the critical signaling pathways are unknown. Applicants have now found that an oncogene, non-receptor cytosolic tyrosine kinase, c-Abl, which is inhibited by imatinib, is critical to the basal and PDGF stimulated elongation of GAG chains on proteoglycans. Imatinib regulates the size of proteoglycans, prevents the increase in proteoglycan size mediated by PDGF, and the size effect is directly due to changes in the size of the GAG chains on proteoglycans and the effect is apparent on the xyloside-initiated GAGs which represent a direct assay of GAG elongation processes. In accord with the “response to retention” hypothesis, a direct corollary of these changes in GAG size are changes in the binding capacity of the proteoglycans for human LDL. Thus, a new therapeutic agent and a key biochemical target for the therapy of atherosclerosis has been identified in this invention.

A “therapeutically effective amount” as used herein is an amount which can cause or effect the desired effect to provide a healthful benefit to the patient. Preferably, the amount will treat and reduce or alleviate the atherosclerosis. The preferred inhibitor, imatinib may be administered in the range of 800 mg (8 capsules) per day or approximately 400 mg/day. More preferably, the concentration will depend on the mode and frequency of administration. However, an effective amount is preferably an amount which can deliver approximately 1 μM of imatinib to the cell or tissue to inhibit c-Abl activation.

Imatinib may be obtained as GLEEVEC™ capsules (imatinib mesylate) from Novartis Pharmaceuticals Corporation.

Toxicity and therapeutic efficacy may be determined by standard pharmaceutical procedures that may involve cell cultures or experimental animals, eg, for determining the LD₅₀ (the dose lethal of 50% of the population) and the ED₅₀ (the dose therapeutically effective in 50% of the population). Data obtained from cell culture assays and animal studies can be used in formulating a range of dosage for use in humans. The dosage of a compound lies preferably within a range of circulating concentrations that include the ED₅₀ with little or no toxicity. The dosage can vary within this range depending upon the dosage form employed and the route of administration used in the method of invention.

For imatinib, details of clinical pharmacology may be obtained from the Novartis Package Insert for GLEEVEC™. Whilst the insert relates to the use of imatinib for the treatment of tumors, details of metabolism and elimination provide guidance to determine suitable therapeutical dosages to establish a therapeutically effective dose for imatinib to treat atherosclerosis.

The c-Abl inhibitors may be administered by any of the following routes, including but not limited to orally, rectally, parenterally (ie intravenously, intramuscularly, or sub-cutaneously), intracistemally, intravascularly, intravaginally, intraperitoneally, topically (as by powders, ointments, or drops), transdermally, bucally, or as an oral or nasal spray.

Although these doses and the regimen described above for treatment of atherosclerosis may be beneficial, it is contemplated that they be considered as guidelines only and that the attending clinician will determine, in his or her judgement, an appropriate dosage and regimen, based on the patient's age and condition as well as the severity of the condition.

In a preferred aspect of the invention, there is provided a method of reducing a risk for atherosclerosis, said method comprising

identifying a risk factor for atherosclerosis; and

administering an amount of a c-Abl inhibitor to the patient to prevent progression of atherosclerosis.

Some patients show a propensity for atherosclerosis. These patients may be identified by showing high risk factors such as high blood pressure, high cholesterol, high lipids and/or diabetes. These factors are closely associated with atherosclerosis. By identifying these patterns early, these patients may be prevented from progressing to atherosclerosis by administering a low dose or an effective dose of a c-Abl inhibitor such as imatinib, which can prevent progression of the disease. The amount may also be used to halt, slow down or maintain the progression and prevent accelerated progression of the disease.

It is envisaged that such amounts will be lower than a therapeutic amount that may be delivered to treat the disease and preferably alleviate or reverse the effects of the disease.

It is also contemplated that the c-Abl inhibitor may be administered in combination with at least one compound which treats a high risk factor associated with atherosclerosis. The compound may be used to treat risk factors such as high cholesterol, high lipid, high blood pressure or diabetes. The compounds may be administered alone or in combination with the c-Abl inhibitor.

Suitable cholesterol lowering drugs include, but are not limited to HMGCOA reductase inhibitors or “statins” including but not limited to atorvastatin, simvastatin, fluvastatin, or pravastatin.

Suitable lipid lowering drugs include but are not limited to peroxisome proliferting activating receptor alpha ligands such as fenofibrate and gemfibrozil (class “fibrates”) and other agents including probucol, or nicotinic acid.

Suitable blood pressure lowering drugs include, but are not limited to, anti-hypertensives including (as classes) calcium antagonists (felodipine etc.), angiotensin converting enzyme inhibitors (enalapril) angiotensin receptor blockers (e.g. irbesartin), diuretics (e.g. indapamide), vasodilators such as prazosine, centrally acting agents such as clonidine, and other agents including methydopa.

Suitable anti-diabetic, anti-hyperglycemic or hypoglycemic agents include, but are not limited to, biguanides (mefformin etc), sulphonylureas, and peroxisome proliferting activating receptor gamma ligands such as glitazones.

It is also with the scope of the present invention that the compounds may further be combined depending on the number of high risk factors the patient displays.

Hence a patient with high cholesterol and high blood pressure may have a corresponding treatment administered along with a c-Abl inhibitor to prevent progression of the atherosclerosis.

In yet another aspect of the present invention, there is provided a method of diagnosing a propensity for atherosclerosis. The method may be based on genetic variations in the concentrations or activities of c-Abl. It is speculated that the degree of activation or availability of c-Abl in a cell may reflect on the GAG mechanisms and GAG length generated in the cell. Such influences on GAG length may affect lipoprotein binding, and hence atherosclerosis.

In yet another aspect of the present invention, there is provided a composition for use in treating atherosclerosis, said composition comprising a therapeutically effective amount of a c-Abl inhibitor or equivalent thereof and a pharmaceutically acceptable carrier.

Preferably, the c-Abl inhibitor is imatinib or equivalent thereof.

The dosage may be of an amount which can deliver a therapeutically effective amount of the c-Abl inhibitor to treat the patient for atherosclerosis, or it may be an amount which prevents further progression of the atherosclerosis. Preferably the dosage will deliver an amount of approximately 1 μM imatinib to the cells.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening,flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, ointments and inhalants. The active compound is mixed under sterile conditions with a pharmaceutically acceptable carrier and any needed preservatives, buffers, or propellants which may be required.

It is also within the scope of the present invention that the composition includes other compounds designed to provide a combined therapy to enhance prevention and treatment of atherosclerosis and other cardiac diseases. For instance, compounds for the treatment of high blood pressure or high cholesterol may be combined with the c-Abl inhibitor. Preferably, the c-Abl inhibitor is imatinib or an equivalent.

Examples of the procedures used in the present invention will now be more fully described. It should be understood, however, that the following description is illustrative only and should not be taken in any way as a restriction on the generality of the invention described above.

### EXAMPLES

### Example 1

### Imatinib and Genistein Inhibit Total Proteoglycan Biosynthesis

(a) Preparation of Human Vascular Smooth Muscle Cells (VSMCs)

Human VSMCs were isolated from otherwise discarded segments of the internal mammary artery from patients undergoing cardiac surgery at the Alfred Hospital (Melbourne, Australia), as described in Nigro J et al. (2002) “Differential effect of gemfibrozil on migration, proliferation and proteoglycan production in human vascular smooth muscle cells”, Atherosclerosis 162(2): 119-129. The Human Ethics Committee of the Alfred Hospital and Baker Heart Research Institute approved the acquisition of tissue.

(b) Imatinib

Imatinib, formerly STI 571 (4-[(4-methyl-1-piperazinyl)methyl]-N-[4-methyl-3-[[4-(3-pyridinyl)-2-pyrimidinyl]amino-phenyl]benzamide methanesulfonate) may be obtained from Novarits Pharma AG, Basle, Switzerland. Genistein (4′,5,7-trihydroxyisoflavone), daidzein(4′,7-dihydroxyisoflavone), human recombinant platelet derived growth factor-BB (PDGF) and transforming growth factor β1 (TGFβ, methyl β-D-xylopyranoside(xyloside), diethylaminoethyl-sephacel (DEAE-sephacel) and cyanogen-bromide-activated Sepharose, were from Sigma Chem. Co. (St. Louis, Mo.). Dulbecco's Modified Eagle Medium (DMEM) containing 5 mM glucose was from GibcoBRL (Grand Island, U.S.A.). Carrier free sodium [³⁵S]sulfate and [³⁵S]Translabel (methionine and cysteine) were from ICN Biomedicals (Irvine, U.S.A.). All other chemicals were of the highest grade commercially available.

(c) Treatment of Smooth Muscle Cell Cultures for the Analysis of Proteoglycan and Glycosaminoglycan Biosynthesis.

Human VSMCs were seeded at a density of 8×10⁴ cells per well on 15 mm diameter Falcon brand plates in DMEM plus 10% serum and followed a 6-day protocol as described in Little, P J et al (2002) “Proteoglycans synthesised by arterial smooth muscle cells in the presence of transforming growth factor-β1 exhibit increased binding to LDLs” Artioscler Thromb Vasc Biol 22:55-60 and Nigro J et al (2002) Atherosclerosis 162(2): 119-129. Serum deprived cells were treated with control media (0.5 ml) containing 0.1% serum and 0.1% DMSO or media containing imatinib (1 μM), genistein (100 μM) or daidzein (100 μM) in the presence or absence of PDGF (50 ng/ml) or TGFβ (2 ng/ml). Cells were metabolically labeled with [³⁵S]-sulfate (50 βCi/well) for the final 24 h.

To study glycosaminoglycan (GAG) synthesis, cells were treated with exogenous xyloside which acts as an artificial and alternative substrate for the enzymes which normally catalyze the initiation of GAG chains on the protein core Nigro J et al (2002)) and Moses, J., Oldberg, A. & Fransson, L. A. Initiation of galactosaminoglycan biosynthesis. Separate galactosylation and dephosphorylation pathways for phosphoxylosylated decorin protein and exogenous xyloside. Eur J Biochem 260, 879-84 (1999)) generating free xyloside-initiated GAG chains. β-D-Xylopyranoside (xyloside) (0.5 mM) was prepared in DMEM with FBS (0.1%) and DMSO (0.1%). The cells were treated with xyloside in the presence of imatinib, genistein and daidzein as described above in the presence and absence of PDGF (50 ng/ml). Cells were metabolically labeled with [³⁵S]-sulfate (50 μCi/well) for 24 hours.

(d) Isolation and Quantitation of Total Proteoglycans, Xyloside-Initiated GAGs and Core Proteins.

The culture media were collected and each plate rinsed with Dulbecco's PBS (0.25 ml). Aliquots (50 μl) of the media sample were spotted in duplicate on 30×15 mm rectangles of Whatman® 3M chromatography paper (Aldrich, Milwaukee, U.S.A.). The paper was washed five times in a solution containing cetylpyridinium chloride (CPC, 1% w/v) and NaCl (50 mM). Following air drying at room temperature, the paper from each sample was placed in liquid scintillation fluid (Packard, Groningen, The Netherlands) and radioactivity counted in a Beckman Coulter liquid scintillation analyzer (Fullerton, U.S.A). Counts from cells labeled with [³⁵S]-sulfate and Tran³⁵SLabel represented total sulfated GAGs and total core protein respectively.

(e) Isolation and Concentration of Proteoglycans and Xyloside-Initiated GAGs.

DEAE-Sephacel columns were prepared as previously described 19 and were washed with low salt buffer containing 8 M urea, 0.25 M NaCl, 0.02 M disodium EDTA and 0.5% Triton X-100. The media of 3 identical treatments was pooled and applied to the column. The columns were washed with low salt buffer to remove non-proteoglycan associated radioactivity. Fractions were collected with high salt buffer containing 8 M urea, 3 M NaCl, 0.02 M disodium EDTA and 0.5% Triton X-100. Fractions containing >15% of the total radioactivity, were pooled and prepared for SDS-PAGE.

Samples were prepared for SDS-PAGE by set counts so that the amount of radioactivity applied to the gel was the same for each treatment. Proteoglycans, cleaved chains and xyloside-initiated GAGs were precipitated using a cold ethanol (95%) solution containing potassium acetate (1.3%) storing at −20° C. for 1 hour and centrifuging as described in Little P J et al (2002) and Nigro J et al (2002)

(e) Cleaving Proteoglycan GAG Chains.

Proteoglycans eluted from DEAE columns and precipitated once with ethanol/potassium acetate solution were treated with sodium borohydride (1 M) in NaOH (50 mM) for 4 h at 45° C. to release the GAG chains. The free GAG chains were precipitated with the ethanol/potassium acetate solution and analyzed for size by SDS-PAGE.

(f) Molecular Sizing of Proteoglycans, Cleaved Chains and Xyloside-Initiated GAG by SDS-PAGE Electrophoresis.

SDS-PAGE gels were prepared following the procedure of Laemelli (Laemmli, U.K. Cleavage of structural proteins during the assembly of the head of bacteriophage T4. Nature 227, 680-5 (1970)) on 4-13% linear gradient separating gels for intact proteoglycans and 4-20% separating gels for cleaved chains and xyloside-initiated GAGs, with a 3.5% stacking gel. For the estimation of apparent relative masses (MᵣS) ¹⁴C high molecular weight standards (GIBCO BRL, Grand Island, U.S.A) were run in separate lanes.

The labeled proteoglycans, cleaved chains, xyloside-initiated GAGs and standard proteins were exposed (3 days) to a Fujifilm imaging plate (Fuji Photo Film Co., Japan) and the imaging plate scanned by the Bio-imaging analyzer BAS-1000 MacBas (Fuji Photo Film Co., Japan).

(g) LDL Affinity Chromatography of Radiolabeled Proteoglycans.

Human blood samples were obtained from healthy subjects. LDL (d=1.019−1.063 g/ml) was purified from blood by sequential density gradient ultracentrifugation as described by Heinecke, J. W., Baker, L., Rosen, H. & Chait, A. Superoxide-mediated modification of low density lipoprotein by arterial smooth muscle cells. J Clin Invest 77, 757-61 (1986). The interaction of total pooled intact proteoglycan preparations with native human LDL was assessed using LDL affinity chromatography, providing a measure of binding capacity. Metabolically labeled culture medium from cells exposed to control media (0.1% DMSO), imatinib (1 μM) and genistein (100 μM) in the presence of PDGF (50 ng/ml) were dialyzed against a solution containing HEPES (10 mM), NaCl (20 mM) and butylated hydroxy toluene (250 μM). Sepharose was prepared in the presence of excess heparin to protect the GAG binding sites. The columns were washed with HEPES buffer above and then with 1M NaCl to remove heparin. Each sample (80,000 cpm) was applied to a separate column. The columns were washed extensively with low salt buffer then eluted (5×0.5 ml) with 1M NaCl to elute bound proteoglycans. Finally the columns were stripped with high salt, high urea buffer. The aggregate radioactivity retained by the LDL column after extensive washing and eluted with 1M NaCl was determined as the binding capacity for the column.

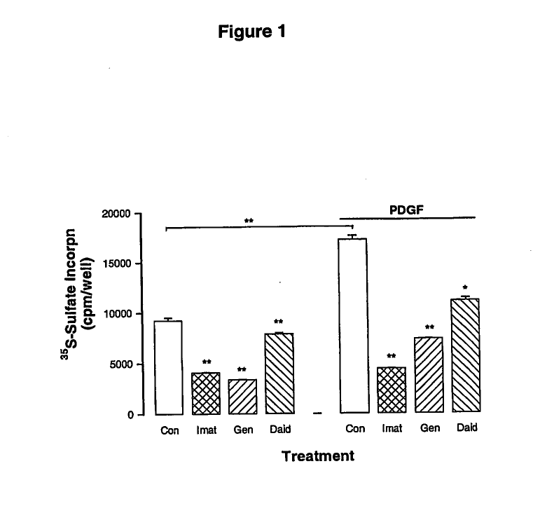

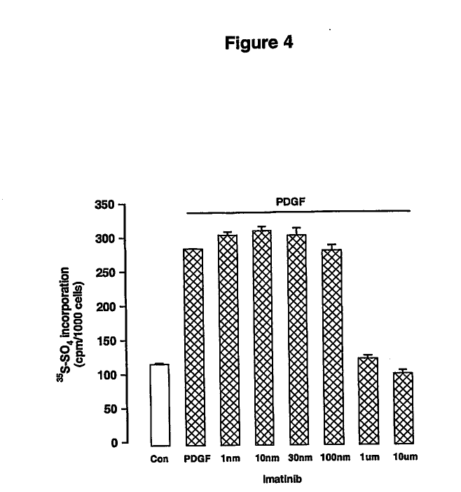

Primate VSMC in culture synthesize and secrete several proteoglycans at an appreciable basal rate which is stimulated by growth factors including PDGF (Schonherr, E., Jarvelainen, H. T., Sandell, L. J. & Wight, T. N. “Effects of platelet-derived growth factor and transforming growth factor-beta 1 on the synthesis of a large versican-like chondroitin sulfate proteoglycan by arterial smooth muscle cells.” J Biol Chem 266, 17640-7 (1991)). Total production is inhibited by genistein suggesting some role for tyrosine kinases in this response (Schonherr, E., Kinsella, M. G. & Wight, T. N. “Genistein selectively inhibits platelet-derived growth factor- stimulated versican biosynthesis in monkey arterial smooth muscle cells”. Arch Biochem Biophys 339, 353-61 (1997). We evaluated the effect of genistein and daidzein, its 5-dehydroxy derivative, which is a less or inactive inhibitor of tyrosine kinases, on total proteoglycan production and compared this with the effect of the new specific tyrosine kinase inhibitor, imatinib which inhibits PDGF receptor and c-Kit receptor tyrosine kinases as well as c-Abl a cytosolic non-receptor tyrosine kinase. Initial dose response curves (data not shown) showed that genistein exhibits maximal effects at 100 μM so this concentration was chosen for experiments with genistein and daidzein. Imatinib exhibits maximum activity at about 1 μM (FIG. 4). Imatinib (1 μM) inhibited basal production by 56% whilst genistein (100 μM) was slightly more active against basal production (64% inhibition) and daidzein showed little inhibitory activity (FIG. 1). PDGF increased the production of proteoglycan by approximately two-fold (P<0.001) and this is due to increases in both the proteoglycan core protein synthesis (most likely versican) and an elongation of the GAG chains on the proteoglycans. The PDGF-stimulated component of proteoglycan production by cells was totally blocked by imatinib (FIG. 1). Genistein was a more potent inhibitor than daidzein but in PDGF treated cells there was some “break through” of the inhibitory effect such that genistein was less inhibitory than imatinib (see FIG. 1). This can be explained by suggesting that inhibition of c-Abl and PDGF receptor activities by imatinib blocks GAG elongation and core protein induction but genistein only inhibits the latter response.

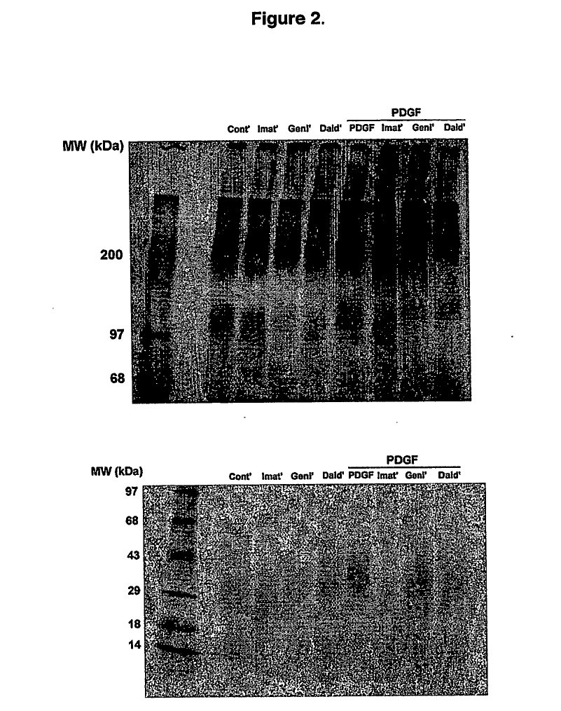

To determine the mechanism by which genistein and imatinib inhibited total proteoglycan biosynthesis we systematically evaluated the effects of the tyrosine kinase pathways on GAG elongation and proteoglycan core protein biosynthesis. Proteoglycans secreted into the media by the human vascular smooth muscle cells were isolated and purified and we examined the size of the proteoglycans by SDS-PAGE. Genistein, and accordingly daidzein, had no effect on proteoglycan size either of the basal proteoglycans or the elongated proteoglycans produced by PDGF treated cells (FIG. 2). In contrast, proteoglycans from cells treated with imatinib (1 μM), showed decreased electrophoretic mobility and thus the size of the bands which correspond to the two smallest proteoglycans produced by these cells namely, decorin and biglycan, were clearly reduced (FIG. 2). We further examined the source of this change in size of the intact proteoglycans by examining specifically the size of the GAG chains chemically released from the proteoglycans by β-elimination. The results considered as the size of the free chains were identical to that for the respective bands of the intact proteoglycans (FIG. 2). Genistein and daidzein had no effect on the size of the GAGs released from proteoglycans produced by untreated cells (FIG. 2, right panel). The effect of PDGF to increase GAG size is clearly apparent (FIG. 2, right panel) and genistein and daidzein had no effect on the elongation of GAG chains stimulated by PDGF. However, inhibiting c-Abl as well as PDGF receptor tyrosine kinase with imatinib, mildly reduced the size of the GAG chains produced under basal conditions and completely prevented the increase in GAG length in PDGF treated cells (FIG. 2, right panel). These data indicted that c-Abl may be a central regulator of GAG elongation.

### Example 2

### Inhibiting c-Abl With Imatinib Reduces the Size of GAG Chains Synthesized on Xyloside.

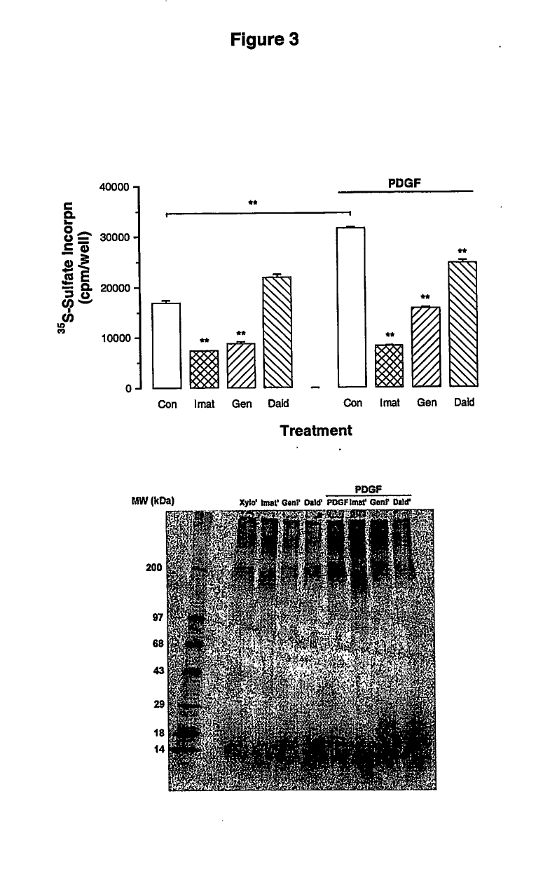

Cell cultures supplemented with xyloside synthesize the normal family of proteoglycans but also free GAG chains which are synthesized on the xyloside acting as a “false acceptor” for GAG initiation. We recently demonstrated that the effect of TGFβ to elongate GAG chains on proteoglycans also occurs for the GAG initiated on xyloside demonstrating that this parameter is controlled independently of core protein synthesis (Little P J et al (2002)). The analysis of the production of the xyloside-initiated GAG chains therefore represents an independent assay of the activity of the GAG elongation process which normally determine the length of GAGs on proteoglycans. The overall quantitative results for the effects of imatinib, genistein and daidzein on the combined production of proteoglycans and GAG chains initiated on xyloside were essentially identical to the effects on total proteoglycans alone noting that the overall rates are increased approximately two fold in cells supplemented with xyloside due to more total GAG synthesis (FIG. 3, upper panel). GAG chains produced on xyloside are considerably smaller than free GAG chains synthesized on and released from proteoglycans (compare FIG. 2 right panel with FIG. 3 lower panel). The band at the bottom of the SDS-PAGE is the GAG chains initiated on xyloside. Genistein and daidzein have no effect on the size of the GAGs either under basal or PDGF stimulated conditions. In contrast, inhibiting c-Abl by treatment of cells with imatinib slightly reduced the size of the GAG chains on xyloside under basal conditions and completely prevented the increase in GAG size in PDGF treated cells. These data demonstrate that imatinib inhibits the processes that lead to elongation of GAG chains on proteoglycans.

### Example 3

### Effect of Tyrosine Kinase Inhibitors on Proteoglycan Core Protein Synthesis.

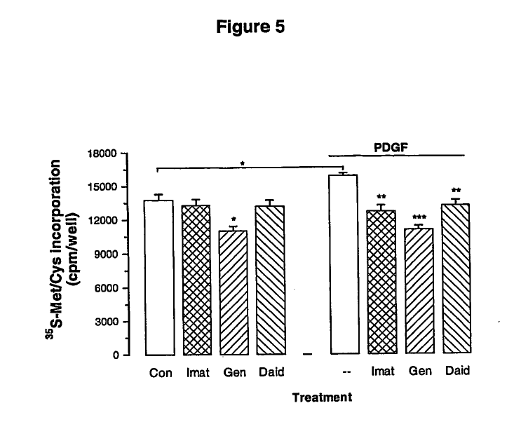

Effects on core protein biosynthesis can be evaluated by metabolically labeling the cells with labeled amino acids (³⁵S-methionine/cysteine) and using an isolation technique “CPC precipitation” which is specific for isolating proteoglycans. Thus, cells were treated identically to the above experiments but labeled with ³⁵S-methionine/cysteine. An earlier report showed that genistein almost completely abolishes the basal expression of versican mRNA and core protein expression in vascular smooth muscle cells and accordingly we observed that the basal rate of proteoglycan core protein biosynthesis was reduced by genistein; daidzein was considerably less active than genistein suggesting that the response was due to inhibition of tyrosine kinases, in particular PDGF receptor tyrosine kinase. Imatinib had a very small but not statistically significant inhibitory effect on basal core protein production. Core protein biosynthesis was stimulated by PDGF (FIG. 5; P<0.001) and this response was attenuated by imatinib, genistein and daidzein (FIG. 5). Thus, imatinib inhibits basal proteoglycan production solely by reducing GAG chain size but in PDGF treated cells it inhibits both the increased expression of core proteins and the GAG elongation process.

### Example 4

### The Reduction of GAG Size by Imatinib Results in Reduced Binding to LDL.

The major potential impact of the finding that an imatinib-sensitive pathway may control GAG length is that considerable evidence indicates that GAG length is a primary determinate of LDL binding affinity. We isolated, purified and dialyzed the proteoglycans secreted into the media by vascular smooth muscle cells activated by PDGF and treated with imatinib and genistein. We used human LDL isolated from normal human plasma to prepare LDL affinity columns in which the potential GAG binding sites were protected during the coupling process by heparin which was subsequently released by treatment of the columns with 1M NaCl. Identical amounts (³⁵S-sulfate 70,000 dpm) of the dialyzed proteoglycan preparations were added to the columns which were washed with low salt buffers to remove non-binding radioactivity and then eluted with 1M NaCl which released proteoglycans bound to the immobilized LDL in the affinity column. We observed a 36% increase in the binding capacity of proteoglycans in PDGF treated cells compared to untreated cells (FIG. 5). The increased binding of proteoglycans produced in PDGF treated cells was completely abolished in imatinib treated cells whereas genistein treatment does not prevent the increased binding stimulated by PDGF. These data indicate that the action of imatinib to reduce GAG chain length has the important functional consequence of reducing the binding capacity to LDL.

### Example 5

### Direct Assays of cAbl Kinase Activity

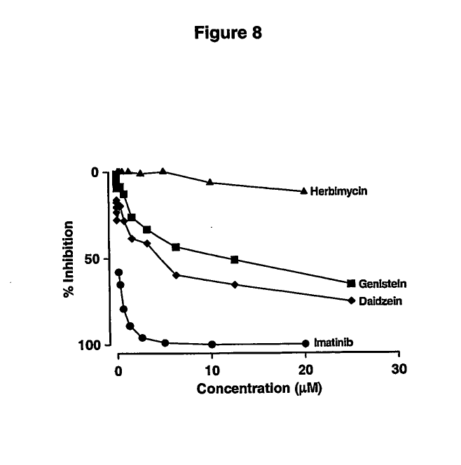

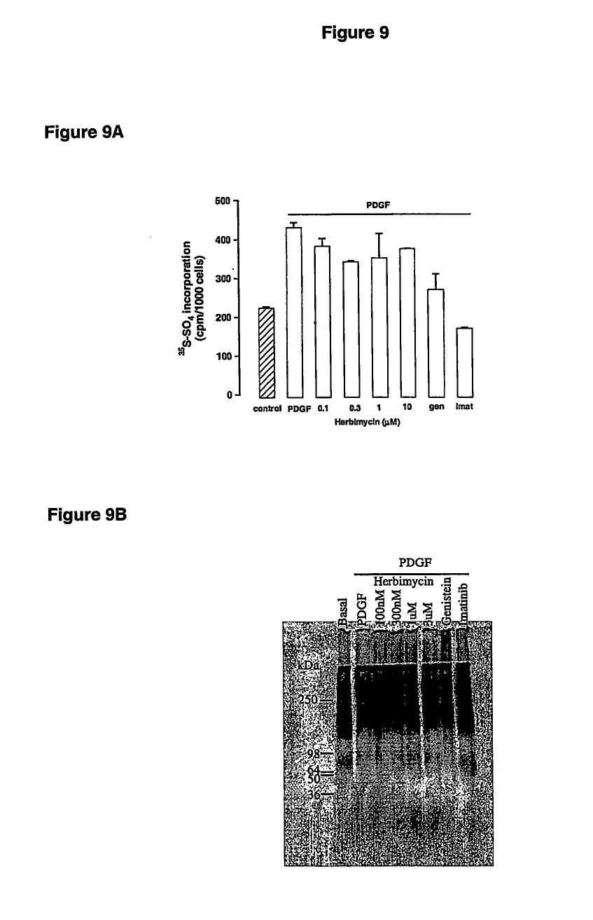

The direct cAbl kinase inhibitory activity of several compounds was evaluated. The direct inhibitory activity of imatinib, genistein, daidzein (an inactive genistein analogue) and another general tyrosine kinase inhibitor, herbimycin was evaluated. Herbimycin is known as a general, irreversible protein tyrosine kinase inhibitor which has specificity for Src tyrosine PTKs. As expected, imatinib shows concentration dependent inhibition of cAbl kinase activity but genistein, daidzein and herbimycin show little activity (FIG. 8). Genistein is 160 times less potent than imatinib confirming that it is not a cAbl inhibitor and supporting the contention that the GAG shortening action of imatinib is mediated via cAbl (inhibition). This data raises the question of the action of herbimycin on GAG length of vascular proteoglycans and it was found that herbimycin does not inhibit radiosulfate incorporation into proteoglycans (FIG. 9A) and accordingly does not reduce the size of GAGs on proteoglycan as evidenced by no change in migratory activity on SDS-PAGE (FIG. 9B). This data is consistent with the fact that the active site of cAbl has a conformation which is distinct from that of Src kinases and little inhibitory cross over between compounds inhibiting cAbl and Src would be expected.

### Example 6

### Inhibition of K562 (cAbl Mediated )Cell Proliferation.

K562 cells are Philadelphia chromosome tumour cells in which constitutively active cAbl (but possibly other pathways) drive the cell proliferation. Inhibition of this cell proliferation determines the anti-leukemic action of imatinib. These cells provide an assay to discriminate inhibition of tyrosine kinases in a whole cell assay.

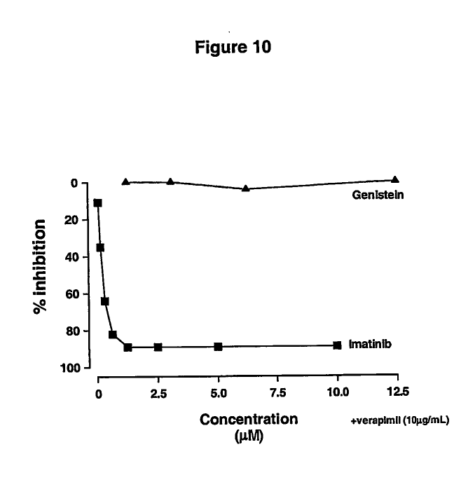

The inhibitory activity of imatinib and genistein on the proliferation of K562 cells was evaluated. Imatinib was a highly potent inhibitor of K562 cells but genistein showed little or no inhibitory activity consistent with a lack of inhibition of cAbl as described above (FIG. 10).

### Example 7

### Extension of Imatinib Action to a New Vascular Bed—Saphenous Vein-Derived Vascular Smooth Muscle Cells.

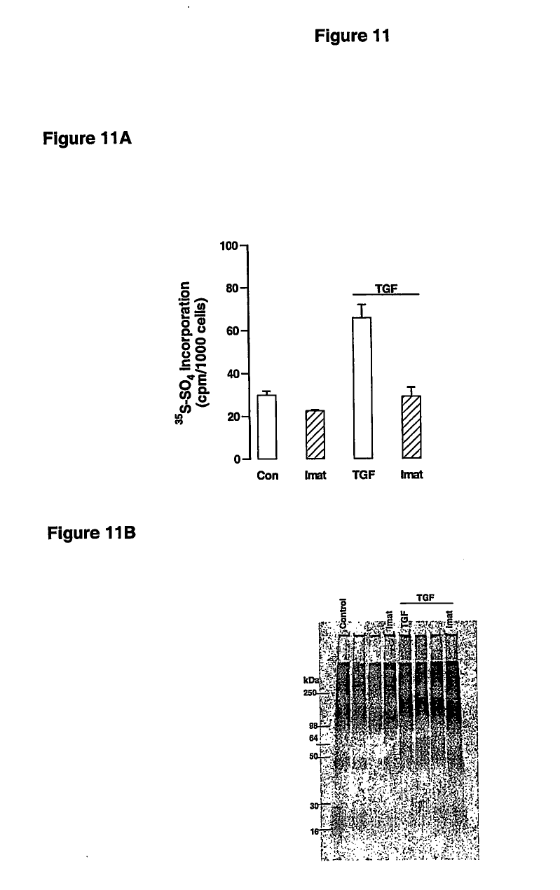

Data provided thus far has demonstrated that imatinib inhibits proteoglycan biosynthesis in internal mammary artery derived vascular smooth muscle cells and shortens the GAG chains. Cultures of vascular smooth muscle cells from human saphenous veins samples obtained (with Ethics approval) from Cardiac Theatres at the Alfred Hospital were prepared. It is now shown that imatinib inhibits radiosulfate incorporation (FIG. 11A) and also shortens GAGs as evidenced by altered mobility of SDS-PAGE. (FIG. 11B). Saphenous vein grafts are more susceptible to atherosclerosis than IMAs when used for by-pass surgery and this data further supports the potential role of cAbl inhibition in preventing LDL binding and retention.

### Example 8

### Further Biochemical Analysis of the GAG Chains on Proteoglycans in Cells Treated with Imatinib.

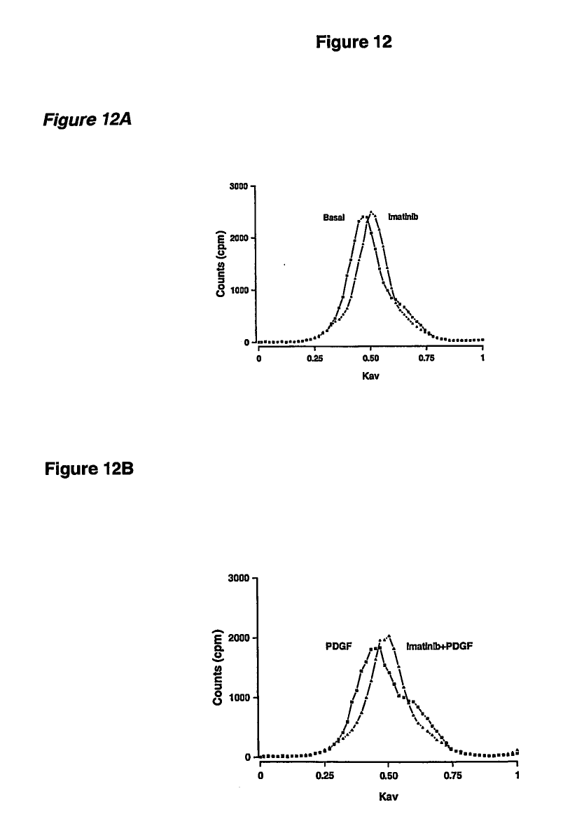

SDS-PAGE has previously been used to demonstrate that cells treated with imatinib (but not genistein) produced smaller proteoglycans, smaller GAGs chains when released from PGs and smaller (artificial) GAG chains made on xyloside as a false acceptor. However, because of the non-classic structure of PGs—ie a core protein with one or more GAG chains, the preferred indeed “gold standard” for the evaluation of the size of these molecules is the use of size exclusion chromatography. Cells have been treated with imatinib and also stimulated with PDGF with and without imatinib. The proteoglycans were isolated and the GAG chains were chemically cleaved for size analysis using Sepharose 6B Size Exclusion Chromatography. This technique separates molecules on the basis of size with larger molecules being excluded from the gel matrix and thus having a shorter transit time and emerging from the column earlier. As expected the chains of PGs released from PGs prepared from cell treated with PDGF were larger than control (Kₐᵥ 0.48 for control v 0.45 for PDGF treated). However, under both control (0.48 v 0.51 (with imatinib)) (FIG. 12A) and PDGF stimulated (0.45 v 0.49 (with imatinib)) (FIG. 12B) conditions PGs produced in the presence of imatinib eluted later than their respective controls indicting that imatinib treatment reduces the size of GAGs.

### Example 9

### Blocking Expression of mRNA for cAbl Using SiRNA

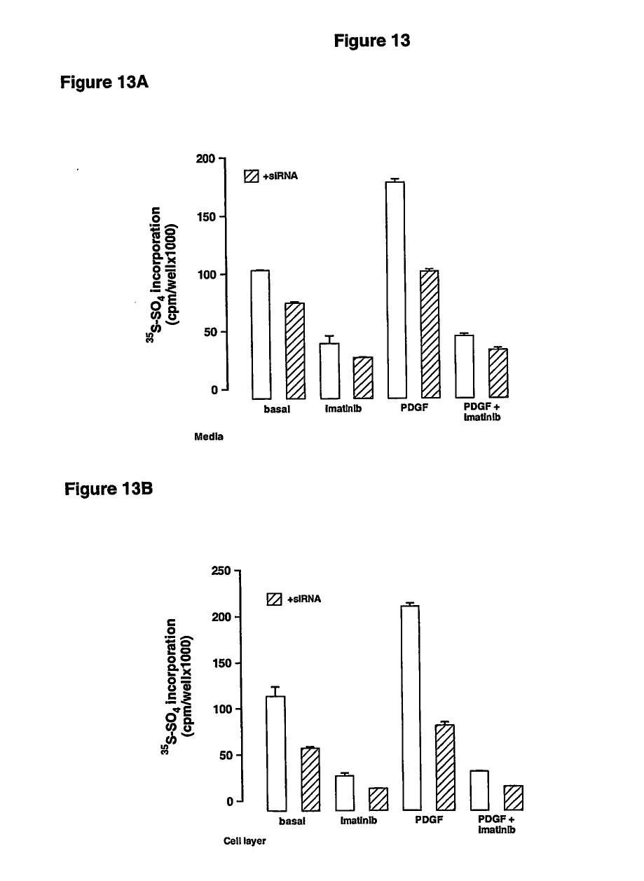

Existing data in earlier examples in the present application has been based on the use of several pharmacological agents to determine that the anti-atherosclerotic effect of imatinib is most likely based on its property to inhibit the non-receptor tyrosine kinase, cAbl. An independent method is available to examine this question by using molecular biology techniques to block the expression of the mRNA for cAbl and thus reduce the actual level of cAbl mRNA, protein and enzyme activity in the cell. A new technique of post-transcriptional gene silencing known as small inhibitory RNA (or siRNA) has recently been developed in which cells are transfected with an siRNA which leads to the breakdown and loss of the target mRNA in this case cAbl RNA. This is a highly specific technique which complements the pharmacological approach. This technique has recently been demonstrated to specifically target the Bcr-Abl mRNA. Target sequences for siRNA have been identified from the scanning of the coding region of the human c-Abl gene. Four of these potential sites were selected and compared to a genome database to identify any sequence homology to other genes. Following identification of target sequences, sense and anti-sense siRNA oligonucleotide templates are constructed consisting of 21 nucleotides encoding the siRNA and 8 nucleotides complementary to a promoter primer. A specific example is target sequence MB 61 which produced the results shown in FIG. 13: Anti-sense siRNA Oligonucleotide template 5′-AAG AAA AAC TTC ATC CAC AGA-3′ and Sense siRNA Oligonucleotide template 5′-AAT CTG TGG ATG AAG TTT TTC-3′. Both sense and anti-sense siRNA oligonucleotide templates were hybridised to a promoter primer. Double stranded siRNA transcription templates was created by extending the hybridised oligonucleotides using the Klenow fragment of DNA polymerase and RNA polymerase used to transcribe the sense and anti-sense siRNA templates which were then hybridised to dsRNA. Vascular smooth muscle cells were transfected after protocol optimisation using GAPDH siRNA.

Several siRNAs were produced and one (“MB 61”) was used to evaluate the effect on radiosulphate incorporation into proteoglycans. The effect of siRNA transfected cells to that of those treated with imatinib was compared. It should be noted (as shown clearly in earlier data) that imatinib inhibits PDGF receptor tyrosine kinase activity (that inhibited by genistein) and cAbl and the inhibition of ³⁵S-sulphate incorporation by imatinib results from GAG shortening and some inhibition of core protein production (the latter being a PDGF receptor signalling outcome). The effect of siRNA transfection to the direct effect of imatinib in control and PDGF targeted cells was compared. ³⁵S-sulphate incorporation was assessed over 24 h using a standard protocol.

Imatinib inhibited radiosulphate incorporation by approximately 60-70% as shown previously and in the siRNA transfected cells the ³⁵S-radiosulphate incorporation was reduced by approximately 30-40% (FIG. 13A). PDGF stimulates ³⁵S-sulphate incorporation into total proteoglycans secreted into the culture media and this is markedly inhibited by imatinib (FIG. 13A). siRNA (MB 61) transfected cells also showed a reduction in ³⁵S-sulphate incorporation of 50%. The effects on the proteoglycans remaining in the cell layer was evaluated and a similar inhibitory effect of the siRNA was observed (FIG. 13B). It must be noted that the siRNA will be more specific than imatinib and the result is consistent with an action of the siRNA to specifically inhibit and reduce the GAG elongating effect of PDGF mediated via cAbl.

The size of the proteoglycans produced in the siRNA transfected cells was evaluated.

### Example 10

### LDL Binding Properties of Proteoglycans Produced by Vascular Smooth Muscle Cells in which cAbl is Inhibited.

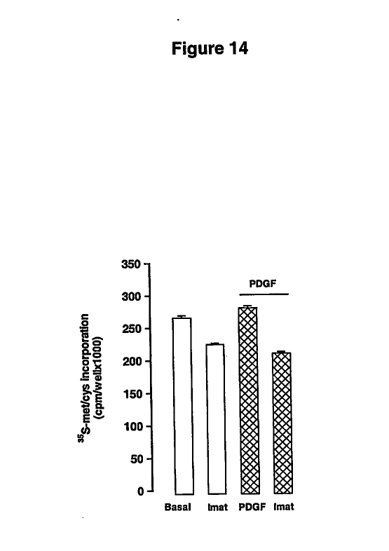

The premise of the present application is that the proteoglycans produced in vascular smooth muscle cells stimulated by atherogenic growth factors (such as PDGF) will show enhanced binding to human LDL and that this will be prevented or reversed if the cells are treated with inhibitors of cAbl such as imatinib. A new approach to these studies is now used in which the proteoglycan core proteins are metabolically radio-labelled and used to study the binding of human LDL. Cells are incubated with Translabel® (ICN Biochemicals) (³⁵S-methioinine and cysteine amino acids) and the amino acids are incorporated into the core proteins. An example of such an experiment is shown in FIG. 14.

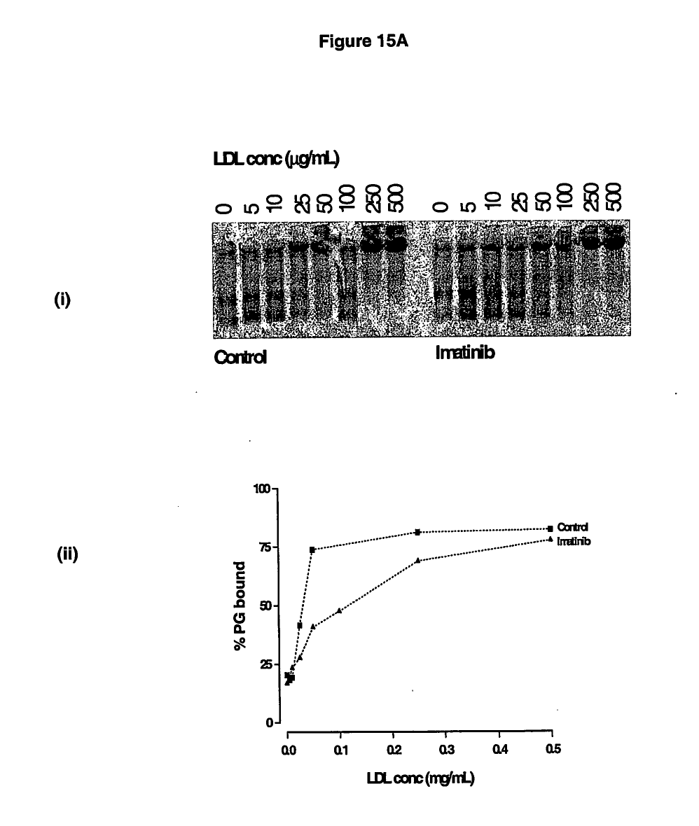

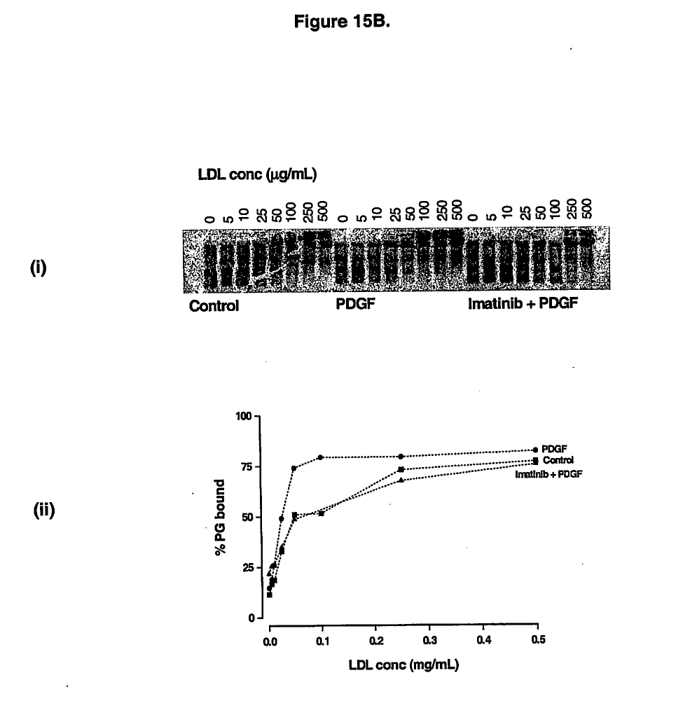

Core protein labelled proteoglycans were isolated by DEAE sepharose ion exchange chromatography and then the binding to human LDL was studied in a Gel Mobility Shift Assay in which a set amount of radiolabeled proteoglycan is incubated with various amounts of LDL and then the bound and free proteoglycans are separated on a flat bed agarose gel. The gel is dried and analysed by phosphoimaging to produced an image (see FIG. 15A, upper panel) and the quantitative data which can be presented and analysed as a binding saturation curve (FIG. 15B lower panel). The effect of inhibiting cAbl with imatinib on the basal and PDGF stimulated proteoglycans produced by vascular smooth muscle cells was studied. Imatinib treatment increased the half-maximal saturation concentration from 35 μg/mL to 90 μg/mL LDL (FIG. 15A lower) indicating a three fold decrease in binding affinity. The binding affinity of proteoglycans produced in PDGF stimulated cells was also reduced when the cells were treated with imatinib. The half-maximal saturation concentration of LDL was 25 μg/mL in PDGF stimulated cells and this was increased to 60 μg/mL in imatinib treated cells clearly indicating that the affinity of binding of proteoglycans to LDL is reduced by imatinib treatment of vascular smooth muscle cells (FIG. 15B). This data clearly demonstrated that the effect of inhibition of cAbl with imatinib is to alter the production and structure of vascular smooth muscle derived proteoglycans in a manner which reduces their binding for human LDL. In an intact blood vessel in an animal this would equate to less LDL binding and retention in the blood vessel and less atherosclerosis.

Finally it is to be understood that various other modifications and/or alterations may be made without departing from the spirit of the present invention as outlined herein.

1. A method of controlling glycosaminoglycan (GAG) chain length in a cell, said method comprising modifying activation of c-Abl in the cell.

2. A method according to claim 1 for reducing GAG chain elongation said method comprising reducing activation of c-Abl in the cell.

3. A method according to claim 2 wherein the activation of c-Abl is reduced by subjecting the cell to a c-Abl inhibitor.

4. A method according to claim 3 wherein the c-Abl inhibitor is imatinib or an equivalent thereof.

5. A method according to claim 4 wherein the cell is subjected to an amount of imatinib or equivalent thereof in the range of 1 nM to 10 μM.

6. A method according to claim 5 wherein the amount of imatinib or equivalent thereof is approximately 1 μM.

7. A method according to claim 1 wherein the cell is selected from the group including an endothelial cell, macrophage, fibroblast and cells associated with atherosclerosis.

8. A method according to claim 7 wherein the cell is an endothelial cell.

9. A method according to claim 7 wherein the cell is a muscle cell.

10. A method according to claim 7 wherein the cell is a vascular smooth muscle cell.

11. A method of controlling lipoprotein binding in a cell or tissue, said method comprising modifying activation of c-Abl in the cell.

12. A method according to claim 11 for reducing lipoprotein binding in a cell or tissue, said method comprising reducing activation of c-Abl in the cell or tissue.

13. A method according to claim 12 wherein the activation of c-Abl is reduced is reduced by subjecting the cell to a c-Abl inhibitor.

14. A method according to claim 13 wherein the c-Abl inhibitor is imatinib or an equivalent thereof.

15. A method according to claim 14 wherein the cell or tissue is subjected to an amount of imatinib or equivalent thereof in the range of 1 nM to 10 μM.

16. A method according to claim 15 wherein the amount of imatinib or equivalent thereof is approximately 1 μM.

17. A method according to claim 11 wherein the cell is selected from the group including an endothelial cell, macrophage, fibroblast and cells associated with atherosclerosis.

18. A method according to claim 17 wherein the cell is an endothelial cell.

19. A method according to claim 17 wherein the cell is a muscle cell.

20. A method according to claim 17 wherein the cell is a vascular smooth muscle cell.

21. A method according to claim 17 wherein the cells are from the neointima or in early artherosclerotic plaques.

22. A method according to claim 13 wherein the inhibitor is bound to a carrier to enhance delivery of the inhibitor to the cell.

23. A method of treating atherosclerosis in a patient, said method comprising administering a therapeutically effective amount of a c-Abl inhibitor or equivalent to the patient.

24. A method according to claim 23 wherein the c-Abl inhibitor or equivalent thereof is imatinib or an equivalent thereof.

25. A method according to claim 23 wherein the c-Abl inhibitor or equivalent thereof is administered to the patient in the range of 400 to 800 mg/day.

26. A method according to claim 23 wherein the c-Abl inhibitor or equivalent thereof is administered by a route selected from the following group including orally, rectally, parenterally, intracistemally, intravascularly, intravaginally; intraperitoneally, topically, transdermally, bucally, or nasally.

27. A method of reducing a risk for atherosclerosis, said method comprising
identifying a risk factor for atherosclerosis in a patient; and
administering an amount of a c-Abl inhibitor to the patient to prevent progression of artherosclerosis.

28. A method according to claim 27 wherein the c-Abl inhibitor or equivalent thereof is imatinib or an equivalent thereof.

29. A method according to claim 27 wherein the c-Abl inhibitor or equivalent thereof is administered in combination with at least one compound which treats a risk factor associated with arthrosclerosis.

30. A method according to claim 27 wherein the patient is identified by showing risk factors selected from the group including high blood pressure, high cholesterol, high lipids and/or diabetes.

31. A method according to claim 29 wherein the compound is a cholesterol lowering drug.

32. A method according to claim 31 wherein the cholesterol lowering drug is an HMGCoA reductase inhibitors or a statin.

33. A method according to claim 32 wherein the statin is selected from the group including atorvastatin, simvastatin, fluvastatin, or pravastatin.

34. A method according to claim 29 wherein the compound is a lipid lowering drug.

35. A method according to claim 34 wherein the lipid lowering drug is selected from the group including peroxisome proliferating activating receptor alpha ligands, probucol, and nicotinic acid.

36. A method according to claim 29 wherein the compound is a blood pressure lowering drug selected from the group including anti-hypertensives, calcium antagonists, angiotensin converting enzyme inhibitors, angiotensin receptor blockers, diuretics, vasodilators, centrally acting agents and methydopa.

37. A method according to claim 29 wherein the compound is an antidiabetic and/or antihyperglydemic and/or hypoglycaemic agent selected from the group including biguanides, sulphonylureas, and peroxisome proliferating activating receptor gamma ligands.

38. A method of diagnosing a propensity for artherosclerosis, said method comprising determining activity of c-Abl in a cell which is suspected of a propensity for atherosclerosis and comparing to a cell which does not have a propensity for atheroclerosis.

39. A composition for treating artherosclerosis said composition comprising a therapeutically effective amount of a c-Abl inhibitor or equivalent thereof and a pharmaceutically acceptable carrier.

40. A composition according to claim 39 wherein the c-Abl inhibitor is imatinib or equivalent thereof.

41. A composition according to claim 39 further including a compound which treats a risk factor associated with artherosclerosis.

42. A composition according to claim 41 wherein the risk factor is selected from the group including high cholesterol, high lipid, high blood pressure or diabetes.

43. A composition according to claim 41 wherein the compound is a cholesterol lowering drug.

44. A composition according to claim 43 wherein the cholesterol lowering drug is an HMGCoA reductase inhibitor or a statin.

45. A composition according to claim 44 wherein the statin is selected from the group including atorvastatin, simvastatin, fluvastatin, or pravastatin.

46. A composition according to claim 41 wherein the compound is a lipid lowering drug.

47. A composition according to claim 46 wherein the lipid lowering drug is selected from the group including peroxisome proliferating activating receptor alpha ligands, probucol, or nicotinic acid.

48. A method according to claim 41 wherein the compound is a blood pressure lowering drug selected from the group including anti-hypertensives, angiotensin converting enzyme inhibitors, angiotensin receptor blockers, diuretics, vasodilators, centrally acting agents, and methydopa.

49. A method according to claim 41 wherein the compound is an antidiabetic and/or antihyperglydemic and/or hypoglycaemic agent selected from the group including biguanides, sulphonylureas, and peroxisome proliferating activating receptor gamma ligands.

50. A method according to claim 13 wherein the c-Abl inhibitor or equivalent thereof is administered in combination with at least one compound which treats a risk factor associated with arthrosclerosis.

51. A method according to claim 23 wherein the c-Abl inhibitor or equivalent thereof is administered in combination with at least one compound which treats a risk factor associated with arthrosclerosis.
